(19) United States
(12) Patent Application Publication
(10) Pub. No.: US 2019/0143094 A1
(43) Pub. Date: May 16, 2019

(54) MODULAR VALVED CONNECTOR FOR DRAINAGE SYSTEMS

(71) Applicant: Merit Medical Systems, Inc., South Jordan, UT (US)

(72) Inventor: John S. DeMeritt, Saddle River, NJ (US)

(21) Appl. No.: 16/186,228

(22) Filed: Nov. 9, 2018

Related U.S. Application Data
(60) Provisional application No. 62/584,411, filed on Nov. 10, 2017.

(51) Int. Cl. A61M 39/10; A61M 27/00; A61M 1/00; A61M 39/24

(52) U.S. Cl. CPC A61M 39/10; A61M 27/00; A61M 1/0088; A61M 2039/1033; A61M 2039/1077; A61M 2210/101; A61M 2205/3344; A61M 39/24

(57) ABSTRACT

Medical devices used to drain a cavity, such as a pleural space of a human body are disclosed. More specifically, the present disclosure relates to a modular valved connector used to connect a drainage catheter, such as a pigtail catheter, to a drainage system that includes a drainage reservoir or receptacle. The modular valved connector may be configured to include a male Luer lock connector.

### RELATED APPLICATIONS

This application claims priority to U.S. Provisional Application No. 62/584,411, filed on Nov. 10, 2017 and titled “Modular Valve Design and Methodology for the Drainage of Bodily Fluids via Non-Tunneled Pigtail Drainage Catheters,” which is incorporated herein by reference in its entirety.

### TECHNICAL FIELD

The present disclosure relates generally to medical devices used to drain a cavity, particularly a pleural space of a human body. More specifically, the present disclosure relates to a modular valved connector used to connect a drainage catheter to a drainage system that includes a drainage reservoir or receptacle.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of a modular valved connector.

FIG. 2 is a side view of the modular valved connector of FIG. 1.

FIG. 3 is a longitudinal, cross-sectional view of the modular valved connector of FIG. 1

FIG. 4A is a longitudinal, cross-sectional view of the modular valved connector of FIG. 1, also depicted with a drainage system connector and a pigtail catheter adapter.

FIG. 4B is a longitudinal, cross-sectional view of the modular valved connector of FIG. 1, assembled with a drainage system connector and a pigtail catheter adapter.

FIG. 5 is an illustration of the modular valved connector of FIG. 1 connected to a pigtail catheter adapter, where the pigtail catheter is inserted into a pleural space, and where the modular valved connector is also connected to a drainage system connector that is coupled to a drainage receptacle.

FIG. 6 is a perspective view of another modular valved connector.

FIG. 7 is a perspective view of the modular valved connector of FIG. 6, also depicted with a pigtail catheter adapter and a drainage system connector.

FIG. 8 is a perspective view of another modular valved connector.

FIG. 9 is a perspective view of the modular valved connector of FIG. 8, also depicted with a pigtail catheter adapter and a drainage system connector.

FIG. 10 is a longitudinal, cross-sectional view of another modular valved connector.

### DETAILED DESCRIPTION

Fluid or air accumulation due to sickness or trauma may develop in areas within a mammalian body not designed to accommodate such accumulation. One particular area prone to abnormal accumulation is between sheets of tissue covering the outside of the lung and lining the chest cavity, known as the pleural space. While a normal functioning pleural space contains approximately 5-20 mL of fluid, fluid turnover occurs on an hourly basis such that approximately 5-10 L of fluid passes through the pleural space every day. Thus, any disruption in fluid turnover may result in an abnormal or over-accumulation of fluid in the pleural space, known as pleural effusion. Gas or air can also abnormally accumulate in the pleural space as the result of certain disease processes as well as the result of trauma including iatrogenic trauma. The abnormal accumulation of air in the pleural space is called a pneumothorax. The abnormal accumulation of both air and fluid in the pleural space is called a hydropneumothorax. The symptoms of a pleural effusion and/or pneumothorax include dyspnea, tachycardia, cough, breathing difficulty, and chest pain as the lungs are prevented from fully expanding upon breathing. Pleural effusions can be caused by a wide variety of acute and/or chronic conditions including pneumonia, congestive heart failure, hypoalbuninemia, kidney disease, pulmonary embolism, pancreatitis, cirrhosis, trauma, complications of open-heart surgery, cancer, and malignancy. Drainage of fluid and/or gas or air in the pleural space is desirable to improve cardiopulmonary function, reduce or eliminate related symptoms, and for diagnostic purposes. This includes acute self-limited conditions such as pneumonia, an exacerbation of a chronic condition such as congestive heart failure, and sometimes-unremitting conditions such as malignant effusions.

There are numerous methods to treat pleural effusion and/or other unwanted fluid accumulation in a mammalian body. Fluid drainage procedures, such as thoracentesis, may be used to provide patient relief. Thoracentesis involves the introduction of a needled catheter into the pleural space through an incision in the skin of the chest wall, and subsequent needle advancement into the chest cavity, after which fluid is drawn out using a syringe or a vacuum source. Drawbacks with this procedure, however, include the fact that the needle may inadvertently puncture the lung, leading to the creation of a pneumothorax from the leakage of air from the injured lung into the pleural space. If the air continues to abnormally accumulate in the pleural space and cannot escape (ball-valve mechanism) it can lead to a tension pneumothorax with cardiovascular collapse, sometimes leading to death. An additional drawback includes the fact that the fluid often re-accumulates in the pleural space after the procedure is performed such that it may become necessary for a patient to undergo the procedure every few days, until the underlying cause can be treated. Percutaneous placed pigtail drainage catheters or surgically placed chest tubes can be placed for the short-term drainage of self-limited or medically treatable pleural effusions (trauma or pneumonia for example); which are both currently invariably attached to large chest tube drainage systems. Pleurodesis, often performed for chronic malignant effusions, is a procedure in which fluid is prevented from accumulating due to the sealing of the space between pleura with either sterile talc or an antibiotic, after first draining the existing fluid. Another method to treat chronic pleural effusions, such as a malignant effusion, is to surgically implant a tunneled chest tube or catheter such that fluid accumulation can constantly or periodically be removed without repeated procedures. The implanted catheter may be connected to an external catheter or drainage tube by a one-way valve mechanism, which allows for intermittent fluid drainage via gravity or through the use of a negative pressure source, such as a vacuum.

Disclosed herein is a universal or modular valved connector that may be used to connect a catheter to a drainage system. Such a system would allow for the intermittent pigtail catheter drainage of pleural fluid without the need for an attached chest tube drainage system, allowing for increased ambulation in the hospital as well as outpatient drainage. The valved connector may include a valve configured to prevent fluid from flowing out of the connector and/or gas or air from flowing into the connector when the connector is in a closed state. The valved connector may also include a proprietary configuration to couple with a proprietary connector at a distal end of the drainage system. The valved connector may also include a proprietary secondary pressure relief valve to allow for the selective venting of positive pressure gas or air in the closed state of the valve in order to treat a pneumothorax, yet at the same time allow for the intermittent and controlled drainage of pleural fluid. Additionally, the valved connector may include a universal or modular portion to couple the valved connector to a standard adapter or hub of a chest tube or drainage catheter. Exemplary drainage systems that can be used and/or coupled with the valved connector disclosed herein include the Aspira Drainage System, the PleurX Drainage System, and/or one or more components of such drainage systems (e.g., connection interfaces, vacuum bottles, pumps, drainage bags, and/or drainage receptacles, etc.). Other drainage systems and/or components that can be employed and/or coupled with the valved connector disclosed herein include those described in U.S. Pat. Nos. 8,337,475, 8,636,721, and 5,484,401, each of which is incorporated herein by reference in its entirety.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use. As specifically applied to the valved connector of a drainage system, the proximal end of the connector refers to the end nearest the drainage reservoir and the distal end refers to the opposite end, the end nearest the drainage device, such as the pigtail catheter. Thus, if at one or more points in a procedure a physician changes the orientation of a valved connector, as used herein, the term “proximal end” always refers to the drainage reservoir end of the connector (even if the distal end is temporarily closer to the physician).

“Fluid” is used in its broadest sense, to refer to any fluid, including both liquids and gases as well as solutions, compounds, suspensions, etc., which generally behave as fluids.

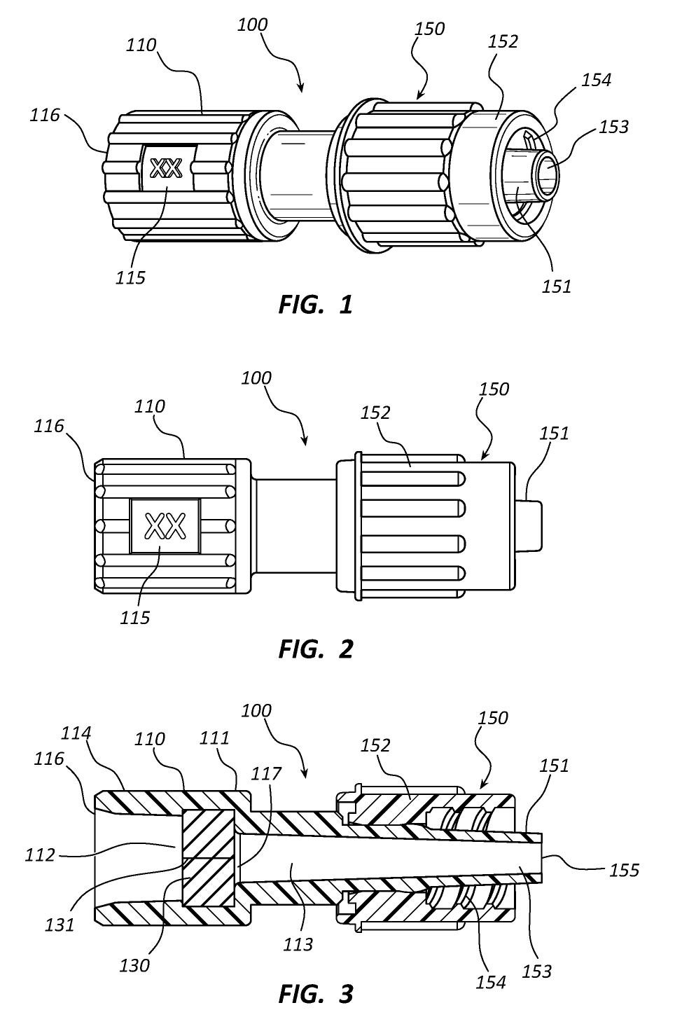

FIGS. 1-10 illustrate different views of several modular valved connectors and related components. In certain views, each device may be coupled to, or shown with, additional components not included in other views. Further, in some views only selected components are illustrated and described to provide detail into the relationship of the components. Additionally, some components may be shown in multiple views, but not discussed in connection with every view. It will thus be understood that the disclosure provided in connection with any figure can be relevant and applicable to disclosure provided in connection with any other figure or embodiment.

FIGS. 1-5 depict one embodiment of a modular valved connector 100. In the illustrated embodiment, the valved connector 100 includes a valve housing 110, a valve mechanism 130, and a connector portion 150.

The valve housing 110 may include a body 111, a valve chamber 112, and a bore 113. The body 111 may be formed from any suitable medical grade thermoplastic material, such as polyethylene, polypropylene, polycarbonate, acrylic, and so forth. A proximal portion 114 of the body 111 may include an external form configured to be grasped by a clinician or patient. The proximal portion 114 may include grip enhancing features, such as longitudinal ribs, bumps, grooves, recesses, and so forth. The body 111 may also include external threads or ears configured to threadingly couple with a medical device, such as a drainage connector.

In some embodiments, the proximal portion 114 may include an indicium 115 disposed on an external surface of the body 111. The indicium 115 may be configured to prevent coupling of the valved connector 100 with medical devices that are not configured or intended to be used for drainage of the pleural space. The indicium 115 may include an emboss of raised lettering or a symbol configured to define the function of the valved connector 100. For example, the emboss may be lettering reading “CHEST VALVE” or “PLEURAL FLUID VALVE.” Another example of the emboss may be a symbol of a lung or an outline of a thoracic cavity. In other embodiments, the indicium 115 may be a coloring of the body 111. For example, the body 111 may be colored pink, which is a color often associated with the lungs. In other embodiments, the body 111 may be colored with any suitable color. The color of the body 111 may be matched with a color-coded band or ring around an adapter or hub of a pigtail catheter to which the valved connector 100 may be coupled.

As illustrated in FIG. 3, the valve chamber 112 is disposed within the proximal portion 114 of the body 111. In other embodiments, the valve chamber 112 may be disposed within any suitable portion of the body 111. As depicted, the valve chamber 112 includes a proximal opening 116 and a distal opening 117 that is in fluid communication with the bore 113. The valve chamber 112 may be configured to retain the valve mechanism 130. The proximal opening 116 may be configured to provide access to the valve mechanism 130 to actuate the valve mechanism 130 when the valved connector 100 is coupled to a drainage connector as will be described below.

FIG. 3 illustrates the valve mechanism 130 disposed within the valve chamber 112. As shown, the valve mechanism 130 comprises a septum 131 disposed at a proximal end of the valve chamber 112 and configured to occlude the proximal opening 116. The valve mechanism 130 is depicted in a closed state where fluid, gas, and air are restricted from flowing out of and into the valved connector 100. The valve mechanism 130 may be configured to be actuated by a connector (and/or a valve actuation member) of a drainage system. When actuated, the valve mechanism 130 may transition from the closed state to an open state (or an actuated state). In the open state the valve mechanism 130 may be configured to allow fluid, gas, and/or air to flow into and out of the valved connector 100.

In other embodiments, the valve mechanism 130 may comprise another suitable form configured to be transitionable from a closed state to an open state. For example, the valve mechanism may be formed as a slit valve, a duckbill valve, an umbrella valve, a compressible valve, and so forth. In some embodiments, the valve mechanism 130 may also include other features and/or functions. For example, the valve mechanism 130 may include a one-way valve configured to allow gas, air, and/or fluid to flow proximally from the valved connector 100 when the valve mechanism 130 is in the open state and to prevent gas, air, and/or fluid from flowing distally into the valved connector 100. In another embodiment, the valve mechanism 130 may include a positive pressure release mechanism (e.g., such as a secondary valve mechanism) in order to treat a pneumothorax and/or prevent the development of a tension pneumothorax in the patient. Continuous positive pressure release of gas or air would be desirable without the simultaneous leakage of fluid from the valve in the closed state, to prevent wetting of the patient. Positive pressure release of gas or air from the device and consequently the pleural space could be accomplished by adding a secondary pressure relief valve (not shown), which could be disposed at a right angle to the primary valve chamber or connector axis. A pressure relief valve could be created by coupling a one-way flow valve (umbrella, duckbill, etc.) with a semipermeable membrane along its inner surface, allowing positive or high-pressure gas or air in the pleural space to escape without the simultaneous leakage of fluid from the device, yet at the same time preventing atmospheric gas or air from entering the system. A suitable membrane for this purpose could be porous polytetrafluoroethylene PTFE, which can be permeable to gas or air but not liquid or water. In yet another embodiment, the valve mechanism 130 may include a dry suction regulator with preset levels of fluid suction. In still another embodiment, the valve mechanism 130 may include an adjustable flow rate controller configured to prevent rapid drainage of fluid from the pleural space to reduce the risk of re-expansion pulmonary edema.

FIGS. 1-3 also illustrate the connector portion 150 of the valved connector 100. In some embodiments, the connector portion 150 includes a male type connector configured to be coupled to a female type connector. In other embodiments, the connector portion 150 comprises a female type connector. The connector portion 150 may also be configured as a modular medical device connector such that the valved connector 100 can be coupled to any medical device having a mating modular medical device connector. For example, in some embodiments, the connector portion 150 may be configured as a male Luer lock connector configured to be coupled to a female Luer lock connector, or a medical device having a female Luer lock connector. For instance, the connector portion 150 may be configured as a male Luer lock connector that is configured to couple to a female Luer lock connector disposed on an end of a catheter, such as a traditional pigtail drainage catheter. In other embodiments, the connector portion 150 may be configured to couple with a uniquely configured mating connector to ensure that the valved connector 100 is used only for an intended purpose, such as draining the pleural space.

It will be appreciated that various types of catheters or drainage catheters can be coupled to the valved connector 100, including commercially available drainage catheters and pigtail drainage catheters. For example, the valved connector 100 can be coupled to tunneled drainage catheters (which can be for long-term use) or non-tunneled drainage catheters (which can be for short-term use), each of which can be inserted into a patient for drainage purposes. It will be further appreciated that any type of catheter having a female Luer lock connector can be coupled to the valved connector 100 in embodiments wherein the valved connector 100 includes a connector portion 150 comprising a male Luer lock connector. The valved connector 100 can also be fixedly attached to a proximal end of a catheter or drainage catheter in certain embodiments.

As shown in FIGS. 1-3, the connector portion 150 is disposed distally of the valve housing 110. The connector portion 150 may be integrally formed with the valve housing 110. In other embodiments, the connector portion 150 may be formed as a separate component and fixedly coupled to the valve housing 110. As illustrated, in some embodiments, the connector portion 150 includes a nozzle 151 and a threaded member 152, such as a threaded nut.

The nozzle 151 may be configured to sealingly and releasably couple with an adapter or hub disposed at a proximal end of a medical device, such as a pigtail catheter. An external surface of the nozzle 151 may be substantially conically shaped (or substantially frustoconically shaped) and distally tapered. The taper angle may be configured to match an internal tapered surface of the adapter or hub such that the valved connector 100 and the adapter or hub of the pigtail catheter are securely coupled. In some embodiments, the taper of the nozzle 151 is configured as a male Luer taper having a 6% taper. A nozzle bore 153 extends through the nozzle 151. The nozzle bore 153 is fluidly coupled to the bore 113 and includes a nozzle opening 155 at a distal end of the nozzle 151. The nozzle bore 153 may be configured to allow fluid, gas, and/or air to flow from the adapter or hub of a pigtail catheter, through the nozzle bore 153 and into bore 113. When the valve 130 is in an open state, fluid, gas, and/or air may further flow into the valve chamber 112.

With continued reference to FIGS. 1-3, the threaded member 152 is disposed around the nozzle 151. The threaded member 152 is shown having a cylindrical form, such as a nut. An external surface of the threaded member 152 may include grip enhancing features, such as longitudinal ribs, grooves, bumps, recesses, and so forth. An internal surface of the threaded member 152 may include threads 154 configured to threadingly couple with external threads or ears of the adapter or hub of the pigtail catheter. The threaded member 152 may be fixedly coupled to a proximal portion of the connector portion 150 such that the whole valved connector 100 may be rotated when coupling to the pigtail catheter adapter or hub. In other embodiments, the threaded member 152 may be configured to rotate around the proximal portion of the connector portion 150 such that only the threaded member 152 may be rotated when coupling the valved connector 100 to the pigtail catheter adapter or hub. In another embodiment, the threaded member 152 may be configured to be longitudinally displaced along a distal portion of the body 111 such that the nozzle 151 may be coupled to the pigtail catheter adapter or hub prior to coupling the threaded member 152 to the adapter or hub.

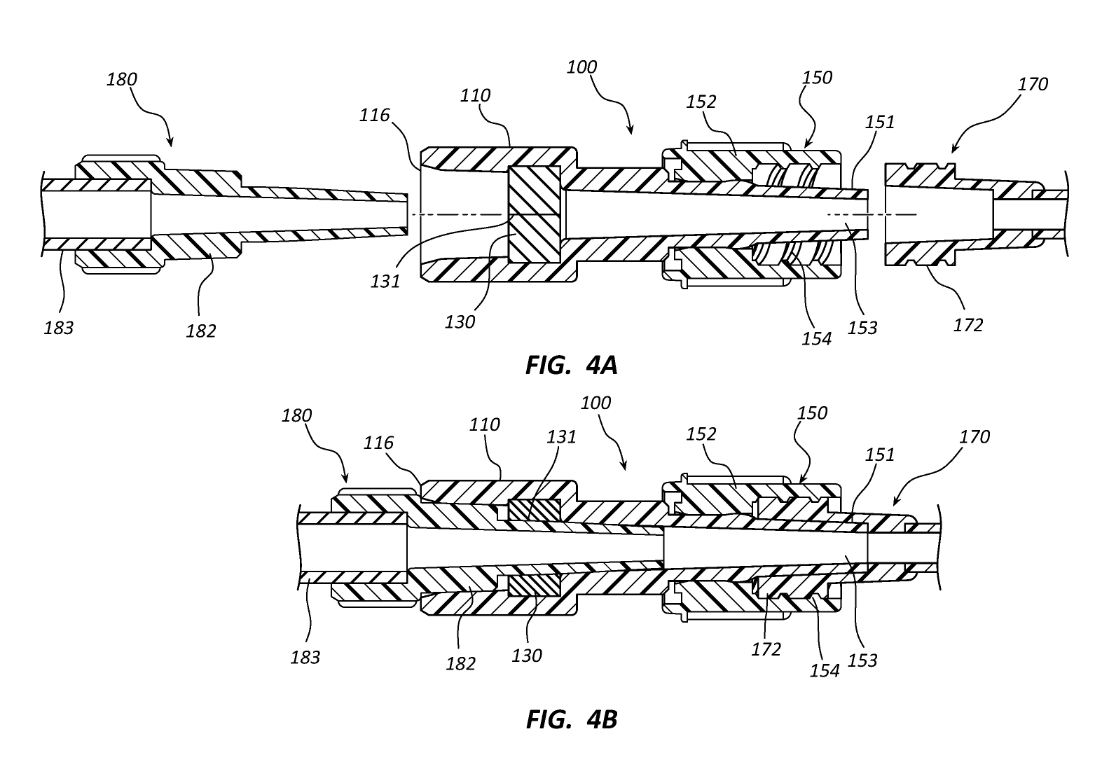

FIGS. 4A-4B depict the valved connector 100 in relationship to a catheter (e.g., a pigtail catheter) 170 and a drainage system connector 180. As illustrated, the valve housing 110 can be coupled to a drainage system connector 180. A valve actuation member (depicted as a tubular protrusion) 182 may be configured to actuate the valve mechanism 130 to allow fluid, gas, and/or air to flow through the valved connector 100. The drainage system connector 180 can be coupled to various types of drainage systems, such as those used in connection with the Aspira Drainage System, the PleurX Drainage System, and those described in U.S. Pat. Nos. 8,337,475, 8,636,721, and 5,484,401, each of which is incorporated by reference in its entirety.

The nozzle 151 and the threaded member 152 can also be coupled to an adapter or hub 172 disposed at a proximal end of a pigtail catheter 170. Various types of adapters 172 can be used, including, but not limited to Luer lock connectors, and/or female Luer lock connectors. As can be appreciated, the distal end of the pigtail catheter 170 can be disposed in a patient's body. It will also be appreciated that the disclosure is not limited to pigtail catheters, and other types of catheters can also be used.

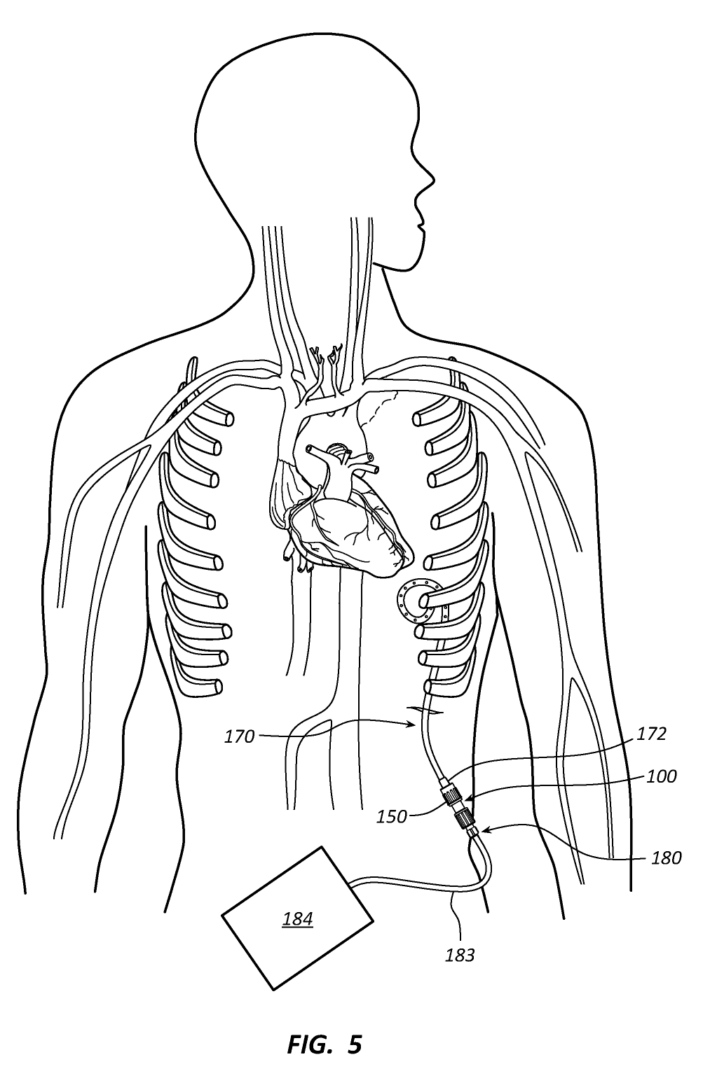

In use, the valved connector 100 may be utilized as a component of a drainage system to withdraw fluid, gas, and/or air from body cavities, such as the pleural space. FIG. 5 illustrates an example of use of the valved connector 100 in a drainage system. As illustrated, a distal portion of the drainage device, such as a catheter (e.g., a pigtail catheter) 170, is percutaneously inserted into the pleural space. A proximal portion of the catheter (e.g., pigtail catheter) 170 extends proximally away from the pleural space. The connector portion 150 of the valved connector 100 is coupled to an adapter or hub 172 disposed at the proximal end of the catheter (e.g., pigtail catheter) 170. A drainage system connector 180 is also coupled to the valve housing 110 such that the valve mechanism 130 may be actuated to allow fluid, gas, and/or air to flow through the valved connector 100. A drainage tube 183 couples the drainage system connector 180 to a drainage reservoir or receptacle 184. In some embodiments, a pump and/or a vacuum source may be coupled to the drainage system to enhance removal of fluid, gas, and/or air from the pleural space. For example, a pump or vacuum source can be coupled at a location that is proximal to the valved connector 100, or at a location that is between the valved connector 100 and the fluid receptacle 184. In other embodiments, a gravity force on the fluid may enhance removal of fluid, gas, and/or air from the pleural space.

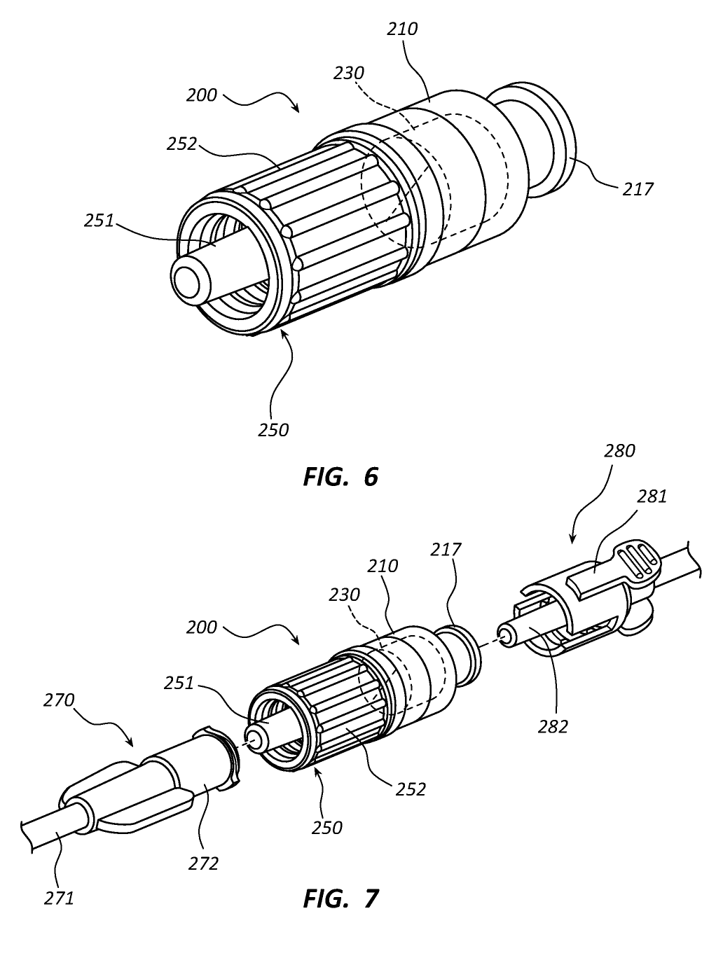

FIGS. 6-7 depict an embodiment of a valved connector 200 that resembles the valved connector 100 described above in certain respects. Accordingly, like features are designated with like reference numerals, with the leading digit incremented to “2.” For example, the embodiment depicted in FIGS. 6-7 includes a connector portion 250 that may, in some respects, resemble the connector portion 150 of FIG. 1. Relevant disclosure set forth above regarding similarly identified features thus may not be repeated hereafter. Moreover, specific features of the valved connector 100 and related components shown in FIGS. 1-5 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the valved connector 200 and related components depicted in FIGS. 6-7. Any suitable combination of the features, and variations of the same, described with respect to the valved connector 100 and related components illustrated in FIGS. 1-5 can be employed with the valved connector 200 and related components of FIGS. 6-7, and vice versa. This pattern of disclosure applies equally to further embodiments depicted in subsequent figures and described hereafter, wherein the leading digits may be further incremented.

FIGS. 6-7 depict another embodiment of a valved connector 200. The valved connector 200 includes a valve housing 210, a valve mechanism 230, and a connector portion 250. The valve housing 210 may include an engagement ring 217 configured to releasably couple with clips 281 of a drainage system connector 280. The clips 281 may retain the valved connector 200 and the drainage system connector 280 in a coupled state until the clips 281 are radially depressed to release the valved connector 200.

The valve mechanism 230 may be disposed within the valve housing 210 and can be configured to prevent flow of fluid, gas, and/or air through the valved connector 200 when in a closed state. The valve mechanism 230 may be configured to be actuated and transition to an open state when the drainage system connector 280 is coupled to the valved connector 200 and a valve actuation member (depicted as a tubular protrusion) 282 couples with the valve mechanism 230.

As illustrated, the connector portion 250 includes a nozzle 251 and a threaded member 252. The nozzle 251 can be configured to sealingly couple with an internal surface of an adapter or hub 272 disposed at a proximal end of a tubing 271 of a catheter 270, such as a pigtail catheter (e.g., a tunneled or non-tunneled pigtail catheter). The connector portion 250 may be configured as a universal medical device connector, such as a male Luer lock connector. The adapter or hub 272 can be configured to mate with the connector portion 250. For example, the connector portion 250 can include a male Luer lock connector, and the adapter hub 272 can include a female Luer lock connector.

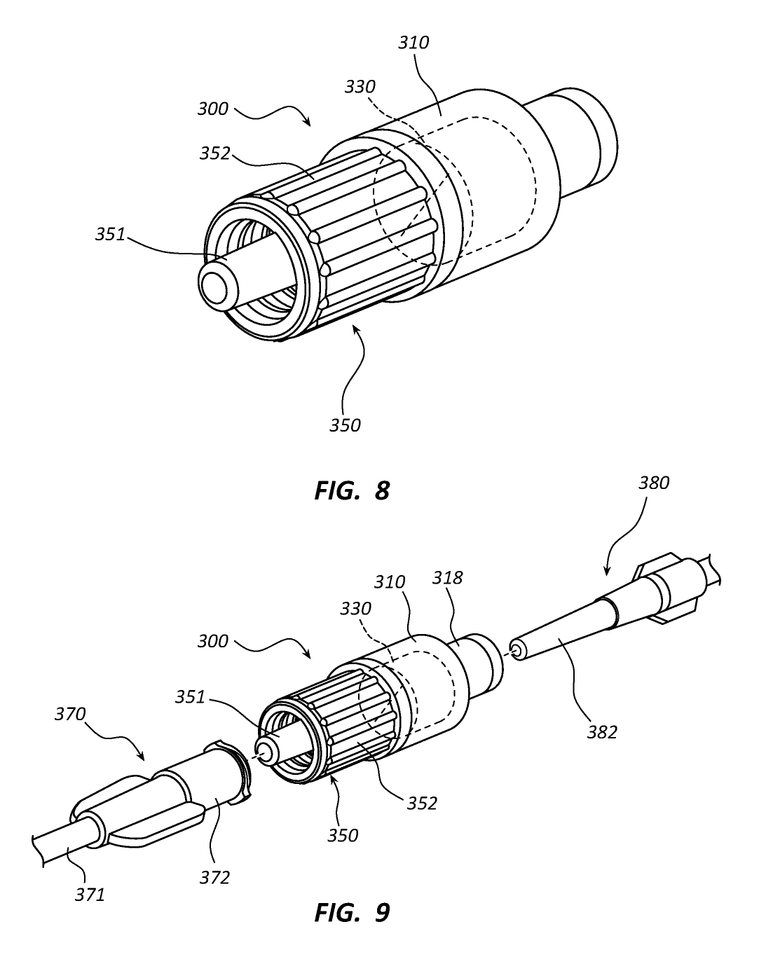

FIGS. 8-9 depict yet another embodiment of a valved connector 300. The valved connector 300 includes a valve housing 310, a valve mechanism 330, and a connector portion 350. The valve housing 310 may include a proximal tubular portion 318 having a proximal opening.

The valve mechanism 330 may be disposed within a distal portion of the valve housing 310 and can be configured to prevent flow of fluid, gas, and/or air through the valved connector 300 when in a closed state. The valve mechanism 330 may be configured to be actuated and transition to an open state when a drainage system connector 380 is coupled to the valved connector 300 such that a valve actuation member (depicted as a tubular protrusion) 382 extends into the proximal tubular portion 318 to actuate the valve mechanism 330.

As illustrated, the connector portion 350 includes a nozzle 351 and a threaded member 352. The nozzle 351 can be configured to sealingly couple with an internal surface of an adapter or hub 372 disposed at a proximal end of a tubing 371 of a catheter 370, such as a pigtail catheter (e.g., a tunneled or non-tunneled pigtail catheter). The connector portion 350 may be configured as a universal medical device connector, such as a male Luer lock connector. The adapter or hub 372 can be configured to mate with the connector portion 350. For example, the connector portion 350 can include a male Luer lock connector, and the adapter or hub 372 can include a female Luer lock connector.

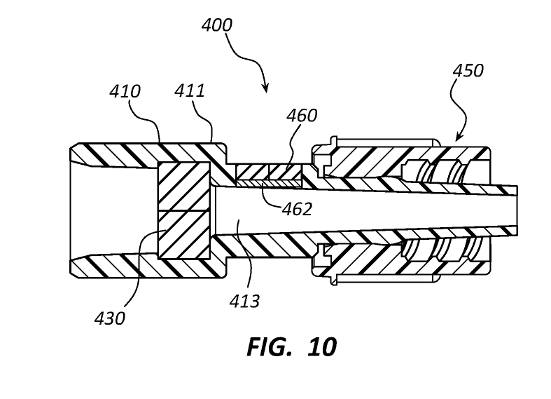

FIG. 10 depicts yet another embodiment of a valved connector 400. The valved connector 400 includes a valve housing 410, a valve mechanism 430, and a connector portion 450. As further shown in FIG. 10, the valved connector 400 includes a secondary valve mechanism 460. In some embodiments, the secondary valve mechanism 460 comprises a pressure release or vent mechanism. For instance, the secondary valve mechanism 460 can be used to treat a pneumothorax and/or prevent the development of a tension pneumothorax in the patient. Further, in some embodiments, the secondary valve mechanism 460 can provide continuous positive pressure release of air or gas (e.g., from the pleural space) while the valve mechanism 430 is in the closed state. The secondary valve mechanism 460 can also be described as being configured to operate independent of the valve mechanism 430.

In certain embodiments, the secondary valve mechanism 460 is disposed in the sidewall of the valve body 411 of the valve housing 410. For example, the secondary valve mechanism 460 can be disposed distal to the valve mechanism 430. The secondary valve mechanism 460 can also be disposed at a location that is between the valve mechanism 430 and the connector portion 450. In particular embodiments, the secondary valve mechanism 460 is disposed at a right angle in relation to the valve mechanism 430 and/or axis of the valved connector 400 (as shown in the illustrated embodiment). Other orientations (e.g., orientation angles and locations) are also contemplated.

In some embodiments, the secondary valve mechanism 460 comprises a vent or a one-way flow valve (e.g., an umbrella valve, a duckbill valve, etc.). The vent or one-way flow valve can allow gas or air to flow out of the system, and prevent or substantially prevent gas or air from flowing into the system. The secondary valve mechanism 460 can also comprise a membrane or semipermeable membrane 462. The membrane 462 can be disposed along an inner surface (as depicted in FIG. 10), or along an outer surface of the valve housing 410. The secondary valve mechanism 460 and/or membrane 462 can be configured to allow the passage or escape of positive or higher-pressure gas or air from the pleural space without allowing (preventing or substantially preventing) the passage or leakage of water or liquid (e.g., pleural or bodily fluid) through the secondary valve mechanism 460 and/or membrane 462. Further, the secondary valve mechanism 460 and/or membrane 462 can be one-way, such that atmospheric gas or air is prevented, substantially prevented, or otherwise prohibited from entering the valve bore 413 and/or system through the secondary valve mechanism 460. An exemplary membrane 462 material that can be used is polytetrafluoroethylene (PTFE), such as porous PTFE, which can be permeable to gas and air but impermeable or substantially impermeable to water and liquid (e.g., pleural or bodily fluid).

Methods of using the modular valved connector are also disclosed herein. In particular, it is contemplated that any of the components, principles, and/or embodiments discussed above may be utilized in a device, a system, or a method of using the same. An illustrative method of draining a pleural cavity, according to one embodiment, includes a step of coupling a modular valved connector to an adapter or hub of a drainage catheter, such as a pigtail pleural drainage catheter. The method can further include a step of coupling a drainage system to the modular valved connector, and a step of applying a negative pressure to the drainage system. In some embodiments, the step of coupling the drainage system to the modular valved connector comprises actuation of a valve mechanism. And in further embodiments, the method comprises drawing fluid from the pleural cavity and collecting the fluid in a drainage receptacle. Other method steps are also contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially impermeable” is recited with respect to a feature, it is understood that in further embodiments the feature can have a precisely impermeable configuration.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A modular valved connector configured to be coupled to a pigtail drainage catheter, comprising:
a valve housing;
a valve mechanism disposed within the valve housing; and
a connector portion configured to be coupled to an adapter of the pigtail drainage catheter.

2. The modular valved connector of claim 1, wherein the valve housing is configured to couple to a drainage system.

3. The modular valved connector of claim 2, wherein the drainage system comprises a valve actuation member configured to actuate the valve mechanism.

4. The modular valved connector of claim 3, wherein the valve actuation member comprises an external coupling mechanism.

5. The modular valved connector of claim 3, wherein the valve actuation member comprises a protrusion configured to compress the valve mechanism.

6. The modular valved connector of claim 3, wherein the valve actuation member comprises an elongate tube configured to pass through the valve mechanism.

7. The modular valved connector of claim 1, wherein the connector portion comprises an internally threaded collar or a male Luer lock connector.

8. The modular valved connector of claim 1, further comprising a secondary valve mechanism and a semipermeable membrane, wherein the semipermeable membrane is permeable to gas and substantially impermeable to bodily fluid.

9. The modular valved connector of claim 1, wherein the connector portion is configured to be sealingly coupled to the adapter of the pigtail drainage catheter.

10. The modular valved connector of claim 1, wherein the valve mechanism is configured to prevent fluid flow when in a closed state and to allow fluid flow when in an open state.

11. The modular valved connector of claim 1, wherein the valve mechanism comprises one or more of a one-way flow valve, a positive pressure release mechanism, a negative pressure release mechanism, and a suction regulator.

12. A pleural cavity drainage system, comprising:
a pigtail drainage catheter comprising an adapter disposed adjacent a proximal end;
a modular valved connector, wherein the modular valve comprises:
a valve housing;
a valve mechanism disposed within the valve housing; and
a connector member configured to couple the modular valved connector to the adapter; and
a drainage system configured to be coupled to the modular valved connector.

13. The pleural cavity drainage system of claim 12, wherein the connector member is configured to sealingly couple to the adapter.

14. The pleural cavity drainage system of claim 12, wherein the connector member comprises an internally threaded collar and a tubular protrusion surrounded by the internally threaded collar.

15. The pleural cavity drainage system of claim 12, wherein the drainage system comprises a coupling member configured to couple the drainage system to the modular valved connector and to actuate the valve mechanism.

16. The pleural cavity drainage system of claim 12, wherein the connector member comprises a male Luer lock connector and the adapter comprises a female Luer lock connector.

17. The pleural cavity drainage system of claim 12, wherein the modular valved connector further comprises a secondary valve mechanism and a semipermeable membrane, wherein the semipermeable membrane is permeable to gas and substantially impermeable to bodily fluid.

18. A method of draining a pleural cavity, comprising:
coupling a modular valved connector to an adapter of a pigtail drainage catheter;
coupling a drainage system to the modular valved connector; and
applying a negative pressure to the drainage system.

19. The method of claim 18, wherein coupling the drainage system to the modular valved connector comprises actuation of a valve mechanism.

20. The method of claim 18, further comprising drawing fluid from the pleural cavity and collecting the fluid in the drainage system.
